(19) United States
(12) Patent Application Publication
(10) Pub. No.: US 2020/0005905 A1
(43) Pub. Date: Jan. 2, 2020

(54) METHOD FOR ASSISTING IN DIAGNOSIS OF THREE MAJOR NEURODEGENERATIVE DISEASES

(71) Applicants: TOAGOSEI CO, LTD, Tokyo (JP); NATIONAL UNIVERSITY CORPORATION NAGOYA UNIVERSITY, Aichi-ken (JP)

(72) Inventors: Makoto Sawada, Nagoya-shi (JP); Nahoko BAILEYKOBAYASHI, Ibaraki-ken (JP); Tetsuhiko YOSHIDA, Ibaraki-ken (JP)

(21) Appl. No.: 16/452,802

(22) Filed: Jun. 26, 2019

(30) Foreign Application Priority Data
Jun. 28, 2018 (JP) 2018-123452

(51) Int. Cl. G16C 20/70; H01J 49/16; H01J 49/00; G16H 50/20; G16H 10/40

(52) U.S. Cl. CPC G16C 20/70; H01J 49/164; G16H 10/40; G16H 50/20; H01J 49/0036

(57) ABSTRACT

The method for assisting diagnosis of three major neurodegenerative diseases consisting of Alzheimer's disease, Parkinson's disease, and ALS provided by the present invention includes:
obtaining a mass spectrum of a specimen collected from a subject by (MALDI/TOF-MS); and
judging the subject to be positive or negative with respect to the three major neurodegenerative diseases on the basis of the magnitude of peak values at a mass-to-charge ratio (m/z) of the resulting mass spectrum of m/z=1733±1 and m/z=2399±1 or a prescribed peak information value derived from the peak values.

### CROSS-REFERENCE TO RELATED APPLICATIONS

The present application claims priority on the basis of Japanese Patent Application No. 2018-123452 filed on Jun. 28, 2018, the content of which is incorporated herein by reference in their entirety.

### TECHNICAL FIELD

The present invention relates to a method for assisting diagnosis of the three major neurodegenerative diseases consisting of Alzheimer's disease, Parkinson's disease and amyotrophic lateral sclerosis by subjecting a specimen (sample) collected from a subject to mass spectrometry.

### TECHNICAL BACKGROUND

With elderly population increased, establishment of methods for diagnosing and treating neurodegenerative diseases are considered to be more important than ever before. Neurodegenerative diseases are neurological disorders in which a specific group of neurons of central nervous system is impaired, resulting in manifestation of symptoms in the manner of decreased cognitive function, ataxia and involuntary movement. In particular, in the present description, the neurodegenerative diseases of Alzheimer's disease, Parkinson's disease and amyotrophic lateral sclerosis (to also be referred to as “ALS”) are defined as the three major neurodegenerative diseases.

Among these three major neurodegenerative diseases, Alzheimer's disease is a neurodegenerative disease in which neurons involved in higher cognitive functions are impaired, and is one of the causes of dementia. Examples of major clinical symptoms of Alzheimer's disease include decreased cognitive functions such as memory impairment, speech impediment or apraxia, personality changes such as violence or abusive language, and abnormal behavior such as wandering.

In addition, Parkinson's disease is a disease of central nervous system involving a loss of neurons in brain that produce dopamine, and examples of symptoms thereof include the onset of uncontrollable shaking on limbs and face, rigidity, slowness of movement (bradykinesia), difficulty in initiating movement, and difficulty in maintaining balance, walking and posture.

In addition, ALS is a progressive neurodegenerative disease in which there is selective inhibition of motor nerves, and typical examples of symptoms thereof include systemic muscular atrophy and muscle weakness (impaired motor functions), spasticity, tendon hyperreflexia, fascicular contraction, gait disturbance, speech impediment (dysarthria), dysphagia, and respiratory disorders.

The status of early symptoms presented by these three major neurodegenerative diseases is difficult to distinguish from other dementia and other diseases causing impairment of motor function, and diagnosis of these diseases currently has to be made by combining the diagnoses of mutually different contents and aspects.

In the case of ALS, diagnosis is made by combining such factors as the presence or absence of clinical symptoms of ALS, rate of advance and exclusion of other diseases causing impairment of motor function. For example, diagnosis is made by suitably combining such tests as a nerve conduction study, electromyography examination, muscle biopsy, nerve image analyses (such as CT or MRI), blood tests, or cerebrospinal fluid examinations.

In addition, in the case of Alzheimer's disease, a comprehensive diagnosis is made on the basis of the results of conducting multiple tests and examinations in the manner of patient interviews, examinations for ascertaining cognitive function (using neuropsychological examinations such as the Mini-Mental State Examination (MMSE)), and image analyses of the brain (such as CT or MRI).

In addition, in the case of Parkinson's disease, a comprehensive diagnosis is made from results obtained by carrying out multiple tests and examinations in the manner of patient interviews, confirmation of neurological symptoms and image analyses of the brain (such as CT or MRI).

In this manner, when diagnosing these three major neurodegenerative diseases, it is necessary to first make diagnosis relating to any of these diseases, and in the case it has ultimately been diagnosed to not be that disease, diagnosis relating to another of these diseases is made until the correct disease has been determined, thereby resulting several different diagnoses being made for each disease. Thus, considerable amount of time and effort are spent until one of these three major neurodegenerative diseases is finally diagnosed, and this is also a cause of delays in the initiation of proper treatment.

For example, even if final diagnosis is left to another diagnostic method, if it were first possible to at least determine which of the three major neurodegenerative diseases is affecting a patient using a single method that is both simple and fast, this would be expected to demonstrate effects such as shortening the subsequent diagnostic process and hastening initiation of treatment. WO 2013/111578 describes a method for assisting diagnosis of Alzheimer's disease that is characterized by detecting the concentrations of Tau protein and amyloid β-peptide in intranasal specimens collected from the nasal cavities of patients. However, diagnosis of the other two major neurodegenerative diseases (Parkinson's disease and ALS) cannot be made with this method.

### SUMMARY OF THE INVENTION

With this in mind, the present invention was conceived in order to solve problems relating to the diagnosis of the three major neurodegenerative diseases, and an object thereof is to provide a method for assisting diagnosis of these diseases that is capable of rapidly diagnosing that a subject is at least afflicted by any of these three major neurodegenerative diseases.

The inventor of the present invention had previously developed a method for accurately analyzing trace components in a sample in a short period of time and with low amount of sample without having to concentrate or pretreat in any way using a type of mass spectrometry of matrix assisted laser desorption/ionization time of flight mass spectrometry (MALDI/TOF-MS). Refer to WO 2015/178249, WO 2017/150680 and WO 2017/150681.

However, in consideration of realizing the above-mentioned object, the inventor of the present invention has analyzed the mass spectra (MS) of the range over which relatively low molecular weight peptides are contained in cerebrospinal fluid (typically such that m/z is about 1000 to 3500) collected from a large number of subjects using a different form of MALDI/TOF-MS differing from the methods described in the Patent Literature. As a result, two statistically significant specific peaks were found that were commonly observed in the cerebrospinal fluid of Alzheimer's disease patients, Parkinson's disease patients, and ALS patients in comparison with those peaks observed when analyzing cerebrospinal fluid derived from healthy subjects, thereby leading to completion of the present invention.

Namely, the method disclosed herein for assisting diagnosis of the three major neurodegenerative diseases consisting of Alzheimer's disease, Parkinson's disease, and ALS includes:
obtaining a mass spectrum of a specimen collected from a subject by MALDI/TOF-MS; and
judging the subject to be positive or negative with respect to the three major neurodegenerative diseases on the basis of the magnitude of peak values at a mass-to-charge ratio (m/z) of the resulting mass spectrum of m/z=1733±1 and m/z=2399±1 or a prescribed peak information value derived from the peak value.

The inventors of the present invention have conducted a statistical study while comparing the mass spectra as determined by MALDI/TOF-MS of a group of specimens (and more specifically, cerebrospinal fluid) collected from multiple healthy subjects with the mass spectra of a group of specimens respectively collected from multiple Alzheimer's disease patients, Parkinson's disease patients, and ALS patients, and have found that the values of two specific peaks commonly observed in the specimens of the Alzheimer's disease patients, Parkinson's disease patients, and ALS patients, namely the values of peaks at m/z=1733±1 and m/z=2399±1, were able to serve as indicators of the diagnosis of the three major neurodegenerative diseases.

Thus, according to the diagnostic assistance method disclosed herein, although not specifically specifying which of these neurodegenerative diseases, a subject can be diagnosed as having high probability of at least suffering from (namely, being positive for) any of these three major neurodegenerative diseases. Consequently, the process for diagnosing whether or not a subject is suffering from any of the three major neurodegenerative diseases can be shortened. In addition, initiation of proper treatment can be hastened on the premise that the subject is suffering from any of these three major neurodegenerative diseases.

In a preferable aspect of the method for assisting diagnosis of the three major neurodegenerative diseases disclosed herein, peak values at m/z=1733±1 and m/z=2399±1 of the above-mentioned resulting spectrum, or prescribed peak information values derived from the peak value, are respectively compared with preliminary prepared corresponding reference values for m/z=1733±1 and m/z=2399±1, and in the case the above-mentioned peak value of m/z=1733±1, or a prescribed peak information value derived from the peak value, exceeds the above-mentioned reference value for m/z=1733±1, and the above-mentioned peak value of m/z=2399±1, or a prescribed peak information value derived from that peak, exceeds the above-mentioned reference value for m/z=2399±1, the above-mentioned subject is judged to be positive for the three major neurodegenerative diseases.

According to the method of this aspect, diagnostic assistance is provided with even higher accuracy regarding the three major neurodegenerative diseases afflicting a subject.

In a preferable aspect of the method for assisting diagnosis of the three major neurodegenerative diseases disclosed herein, the specimen collected from the above-mentioned subject is characterized as being cerebrospinal fluid.

Cerebrospinal fluid has little contaminants, allows acquisition of a favorable mass spectrum by MALDI/TOF-MS, and can realize a judgement of whether a subject is suffering from the three major neurodegenerative diseases with higher accuracy or not.

### DESCRIPTION OF THE RELATED EMBODIMENTS

The following provides an explanation of preferred embodiments of the present invention. Matters other than those matters specifically mentioned in the present description (such as those substances that form peaks at m/z=1733±1 and m/z=2399±1 as determined by MALDI/TOF-MS disclosed herein) that are required for carrying out the present invention (such as general matters relating to the method used to perform mass spectrometry on a specimen using MALDI/TOF-MS, the method used to collect specimens from subjects, or the method used to prepare samples for use in MALDI/TOF-MS) can be understood to be design matters for people with ordinary skill in the art based on conventional technology in fields such as mechanical engineering, computer science, cellular engineering, physiology, medicine, pharmacology, organic chemistry, biochemistry, genetic engineering, protein engineering, molecular biology or genetics. The present invention can be carried out based on the contents disclosed in the present description and common general technical knowledge in the art.

In the present description, a “healthy subject” indicates a healthy person who is completely absent of clinical symptoms of the three major neurodegenerative diseases and has not been diagnosed with any of the three major neurodegenerative diseases. Meanwhile, in the case of referring to a “patient” as related to the three major neurodegenerative diseases indicates an individual who is suffering from at least one of the three major neurodegenerative diseases according to a diagnostic process established in the prior art other than the diagnostic assistance method disclosed herein.

In addition, in the present description, in the case of indicating “M±1” as related to the mass-to-charge ratio (m/z) M in a mass spectrum determined by MALDI/TOF-MS, the “±1” refers to the range of error that can occur according to the analysis apparatus, analysis method, measurement conditions and differences present therein. Here, although the range of error herein has been set to “±1” based on the range of error that can occur in mass spectrometry using general-purpose MALDI/TOF-MS, this range of error is not limited thereto, but rather may be suitably set (such as to ±0.5 or ±2) corresponding to the analysis apparatus, analysis method and measurement conditions provided it enables identification of peak values (typically, peak intensity in % Int.) characteristic of the three major neurodegenerative diseases.

The method for assisting diagnosis of the three major neurodegenerative diseases disclosed herein is a method for determining mass spectra over the range in which m/z is about 1000 to 3500 by MALDI/TOF-MS and assisting diagnosis of the three major neurodegenerative diseases based on whether or not the above-mentioned two statistically significant specific peaks are observed, and can use specimens in various states provided the method can be carried out accurately. The specimen collected from a subject is preferably cerebrospinal fluid. Since cerebrospinal fluid is a clear liquid having few cellular components or other contaminants, it is preferable as a sample used in MALDI/TOF-MS. However, other specimens such as serum may also be used without being limited to cerebrospinal fluid.

MALDI/TOF-MS carried out in the method for assisting diagnosis of the three major neurodegenerative diseases disclosed herein can be preferably carried out based on the instructions and so forth provided corresponding to the apparatus used. A typical example of a mass spectrometry apparatus preferable for carrying out the present invention is a member of the AXIMA (registered trademark) series manufactured by Shimadzu Corporation. For example, MALDI/TOF-MS can be preferably carried out under conditions employed in analyzing a peptide component having a comparatively low molecular weight collected from a biological sample (namely, cerebrospinal fluid or other body fluid).

Preferable examples of the matrix include substances suitable for low to medium molecular weight samples such as a-cyano-4-hydroxycinnamic acid (CHCA), 2-(4-hydroxyphenylazo) benzoic acid (HABA) or 2,5-dihydroxybenzoic acid (gentisic acid, DHBA). Mass spectra can be measured at a suitable laser intensity using a mode enabling measurement of positive ions (such as a linear mode) for the measurement mode.

In the method for assisting diagnosis of the three major neurodegenerative diseases disclosed herein, a subject can be judged to be positive or negative for the three major neurodegenerative diseases based on the magnitude of peak values at m/z=1733±1 and m/z=2399±1 in a mass spectrum obtained for that that subject.

In general, these two peak values (typically, peak intensities in % Int.) obtained from healthy subjects are of a considerably lower level (or are not observed as peaks during data analysis) in comparison with the peak values of the same m/z obtained from patients. Thus, by setting a preset peak value for healthy subjects (which can have a peak intensity (% Int.) of 0 or 5 or less) as a reference value, cases in which the peak values of a subject is higher than that reference value can be judged as being positive for the three major neurodegenerative diseases, which cases in which the peak values are lower can be judged as being negative.

Alternatively, a subject may be judged to be positive or negative for the three major neurodegenerative diseases employing various peak information values derived from peak values at m/z=1733±1 and m/z=2399±1 in a mass spectrum obtained for that subject.

For example, the ratio between the peak value (peak intensity in % Int.) and peak values (peak intensity in % Int.) at m/z=1733±1 and m/z=2399±1 for any reference substance for which m/z differs may also be used as a data value derived based on the above-mentioned peak values. For example, a prescribed amount of reference substance (such as a type of peptide or other organic materials having a known molecular weight that is not present in body) may be added to a subject sample, and reference values may be respectively set for the values of X/A and Y/A as the ratios of a peak value X at m/z=1733±1 and a peak value Y at m/z=2399±1 to a peak value A of the measured reference substance.

Alternatively, multiple laser irradiation may be carried out, and an integrated value obtained integrating a peak value equal to or greater than a prescribed peak value (for example, having peak intensity (% Int.) of 0 or 5 or less) for each mass spectrum obtained each time corresponding to each laser irradiation, or an integrated value (% Cont.) obtained by integrating the number of times that the peak was detected, can be used as a peak information value for assisting diagnosis of the three major neurodegenerative diseases.

Although the following provides an explanation of a preferable aspect relating to the method for assisting diagnosis of the three major neurodegenerative diseases disclosed herein, this is not intended to limit the present invention to the aspect explained here.

Healthy subjects (n=21), Alzheimer's disease patients (n=55), Parkinson's disease patients (n=10), and ALS patients (n=12) were selected as subjects and cerebrospinal fluid was collected from each subject to prepare a total of 98 specimens. Cerebrospinal fluid is clear liquid having few contaminants and can be used directly for MALDI/TOF-MS without carrying out any complex pretreatment.

First, cerebrospinal fluid (specimen) and matrix solution were mixed at a volume ratio of 1:1. Solution containing CHCA at a concentration of 5 mg/mL in a 50% by volume aqueous acetonitrile solution containing 0.1% by volume of trifluoroacetic acid (TFA) (0.1% TFA/50% aqueous ACN solution) was used for the above-mentioned matrix solution. 2 μL of the cerebrospinal fluid sample obtained mixing the matrix solution with cerebrospinal fluid (specimen) were dropped into 384-well plate for MALDI/TOF-MS followed drying to immobilize the sample on the plate.

The AXIMA (registered trademark) Performance model manufactured by Shimadzu Corporation was used for the mass spectrometry apparatus (MALDI/TOF-MS). Measurement conditions were as indicated below.

Laser light source: N₂ sealed laser (λ=337.1 nm)
Accelerating voltage: +20 kV
Reflectron mode: Linear mode

Furthermore, Angiotensin II (m/z=1046.54), ACTH fragments 18-39 (m/z=2465.20) and insulin (m/z=5730.61) were used as calibrants (calibration standards) and the measuring instrument was calibrated according to the external standard method. Each cerebrospinal fluid sample was irradiated with laser light to obtain a mass spectrum thereof

Peak values (% Int.) at each m/z detected in the resulting mass spectra (only those in which total ion current fell within the range 20,000,000 to 30,000,000 were used) were then integrated to obtain the peak information value at each m/z. This processing was carried out for each specimen collected from healthy subjects (n=21), Alzheimer's disease patients (n=55), Parkinson's disease patients (n=10), and ALS patients (n=12).

The presence of statistically significant difference was respectively tested between each patient group and the healthy subject group using the resulting peak information values of each group. The Mann-Whitney U-test was used here in a two-sided test. The above-mentioned MALDI/TOF-MS procedure and U-test were respectively carried out twice for each group.

As a result of this test, m/z having a particularly small P value indicating level of significance, or in other words, m/z exhibiting a particularly high significant difference between each patient group and the healthy subject group were identified. Five m/z indicating a particularly high significant difference between each patient group and the healthy subject group are shown in the following Table 1. In the table, AD, PD, and ALS indicate results for the Alzheimer's disease patient group, Parkinson's disease patient group, and ALS patient group, respectively.

TABLE 1

| Patient | | P value according to U-test | | |
|---|---|---|---|---|
| group | m/z | First run | Second run | Average |
| AD (1) | 2391.22 | 0.0001 | 0.0009 | 0.0005 |
| AD (2) | 1718.57 | 0.0045 | 0.0003 | 0.0024 |
| AD (3) | 1733.52 | 0.0047 | 0.0048 | 0.0047 |
| AD (4) | 2399.39 | 0.0072 | 0.0028 | 0.0050 |
| AD (5) | 2259.52 | 0.0083 | 0.0019 | 0.0051 |
| PD (1) | 2027.95 | 0.0021 | 0.0072 | 0.0047 |
| PD (2) | 1733.52 | 0.0003 | 0.0109 | 0.0056 |
| PD (3) | 1601.14 | 0.0026 | 0.0103 | 0.0065 |
| PD (4) | 2162.80 | 0.0118 | 0.0016 | 0.0067 |
| PD (5) | 2399.90 | 0.0058 | 0.0139 | 0.0098 |
| ALS (1) | 1733.52 | 0.0066 | 0.0038 | 0.0052 |
| ALS (2) | 1454.95 | 0.0017 | 0.0108 | 0.0063 |
| ALS (3) | 2399.90 | 0.0038 | 0.0093 | 0.0066 |
| ALS (4) | 1443.94 | 0.0070 | 0.0155 | 0.0112 |
| ALS (5) | 1585.91 | 0.0202 | 0.0026 | 0.0114 |

As shown in Table 1, peaks at m/z=1733±1 and m/z=2399±1 demonstrated high significant difference in the mass spectra of any of the AD, PD, and ALS patient groups in a comparison with the healthy subject group.

Thus, assistance in judging subjects to be positive or negative for the three major neurodegenerative diseases can be provided based on the magnitude of peak values at m/z=1733±1 and m/z=2399±1 or prescribed peak information values derived from these peak values.

Moreover, conducting further studies as to whether or not there are peaks having conspicuous m/z indicated in rows AD (1), PD (1), and ALS (2) listed in Table 1, further assistance can be provided for judging whether or not subjects judged to be positive for the three major neurodegenerative diseases are suffering from any one of Alzheimer's disease, Parkinson's disease, and ALS or are concomitantly suffering from two or more thereof

As described above, according to the diagnostic assistance method disclosed herein, a subject can be judged to have a high probability (namely, be positive) of at least suffering from any of the three major neurodegenerative diseases. Consequently, the process for diagnosing whether or not a subject is suffering from any of the three major neurodegenerative diseases can be shortened. In addition, the initiation of proper treatment can be hastened on the premise that a subject is suffering from any of the three major neurodegenerative diseases.

1. A method for assisting diagnosis of the three major neurodegenerative diseases consisting of Alzheimer's disease, Parkinson's disease, and amyotrophic lateral sclerosis (ALS), comprising:
obtaining a mass spectrum of a specimen collected from a subject by matrix assisted laser desorption/ionization time of flight mass spectrometry (MALDI/TOF-MS), and
judging the subject to be positive or negative with respect to the three major neurodegenerative diseases on the basis of the magnitude of peak values at a mass-to-charge ratio (m/z) of the resulting mass spectrum of m/z=1733±1 and m/z=2399±1 or a prescribed peak information value derived from the peak values.

2. The diagnostic assistance method according to claim 1, wherein peak values at m/z=1733±1 and m/z=2399±1 of the resulting spectrum, or prescribed peak information values derived from the peak values, are respectively compared with preliminary prepared corresponding reference values for m/z=1733±1 and m/z=2399±1, and
in the case where the peak value of m/z=1733±1, or a prescribed peak information value derived from the peak value, exceeds the reference value for m/z=1733±1, and the peak value of m/z=2399±1, or a prescribed peak information value derived from the peak value, exceeds the reference value for m/z=2399±1, the subject is judged to be positive for the three major neurodegenerative diseases.

3. The diagnostic assistance method according to claim 1, wherein the specimen collected from the subject is cerebrospinal fluid.

4. The diagnostic assistance method according to claim 2, wherein the specimen collected from the subject is cerebrospinal fluid.
